(19) United States
(12) Patent Application Publication
(10) Pub. No.: US 2014/0199349 A1
(43) Pub. Date: Jul. 17, 2014

(54) PH-SENSITIVE HYALURONIC ACID DERIVATIVE AND USES THEREOF

(71) Applicants: Jean-Dean Yang, Dayuan Township (TW); Ayi-Man Chou, Taichung City (TW); Jui-Hsiang Chen, Hsinchu City (TW); Hsia-Wei Liu, Taipei City (TW); Shian-Jy Wang, Jhudong Township (TW); Tse-Min Teng, Ji'an Township (TW); Hsiang-Fa Liang, Hsinchu City (TW); Ming-Cheng Wei, Yangmei City (TW); Maggie J.M. Lu, Jhudong Township (TW)

(72) Inventors: Jean-Dean Yang, Dayuan Township (TW); Ayi-Man Chou, Taichung City (TW); Jui-Hsiang Chen, Hsinchu City (TW); Hsia-Wei Liu, Taipei City (TW); Shian-Jy Wang, Jhudong Township (TW); Tse-Min Teng, Ji'an Township (TW); Hsiang-Fa Liang, Hsinchu City (TW); Ming-Cheng Wei, Yangmei City (TW); Maggie J.M. Lu, Jhudong Township (TW)

(73) Assignee: INDUSTRIAL TECHNOLOGY RESEARCH INSTITUTE, Hsinchu (TW)

(21) Appl. No.: 14/128,376

(22) PCT Filed: Dec. 29, 2011
(86) PCT No.: PCT/CN2011/084936

§ 371 Date: Dec. 20, 2013

(51) Int. Cl. A61K 47/36; A61K 9/107; A61K 31/704

(52) U.S. Cl. CPC A61K 47/36; A61K 31/704; A61K 9/1075
USPC 424/400; 525/54.2; 525/54.1; 514/772.1; 514/34

(57) ABSTRACT

Disclosed is a pH-sensitive hyaluronic acid derivative, comprising at least one repeat unit as shown in the following formula (I), wherein HA represents a unit comprising N-acetyl-D-glucosamine and D-glucuronic acid, q represents an integer of 2 to 10,000; A represents a biocleavable linkage comprising at least one of hydrazone, acetal, ketal and imine; M represents at least one of a hydrophobic fragment, a hydrophilic fragment, and an amphiphilic fragment, and p represents the number of [A-M] directly grafted onto each HA unit, p represents an integer of 0 to 4, and the p of each HA unit is not 0 at the same time.

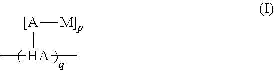

### TECHNICAL FIELD

The present invention relates to a hyaluronic acid derivative and uses thereof, in particular relates to a pH-sensitive hyaluronic acid derivative.

### BACKGROUND

Hyaluronic acid is a linear mucopolysaccharide consisting of repeated units of N-acetyl-D-glucosamine and D-glucuronic acid. Hyaluronic acid was first discovered in the vitreous body of bovine eyes. Afterward, hyaluronic acid has also been found in other tissues, such as the extracellular matrix (ECM) or synovial fluid. The biological function of hyaluronic acid is primarily for cell protection and lubrication, modulation of cell movement on the viscoelastic matrix, stabilization of the collagen network structures and protection of the collagen network structure from mechanical damage.

The cell receptor on the cell surface against hyaluronic acid is CD44. The binding of hyaluronic acid and CD44 enhances cell aggregation, movement, proliferation, activation and activities among cells and the adhesion therebetween. The bonding of hyaluronic acid and CD44 enhances epithelial-mesenchymal transition (EMT) of cancerous cells, causing cancerous cells invading circulatory systems and lymphoid systems and binding to originally normal cells, and, finally, resulting in cancer metastasis.

The formation of micelles by using hyaluronic acid has been studied. When the concentration is higher than the critical micelle concentration (CMC), according to thermodynamics, hyaluronic acid is going to self-assemble in an aqueous matrix to form nano-scaled micelles. Several articles have disclosed the formation of micelles and encapsulated drugs with hyaluronic acid, for example, U.S. Pat. No. 6,350,458B1 or No. 7,780,982B1.

U.S. Pat. No. 6,350,458B1 has disclosed a composition comprising at least one micelle-forming material, macromolecular pharmaceutical agent-like hormones or antibodies, alkali metal alkyl sulfate, alkali metal salicylate and pharmaceutically acceptable edetate, for delivering the macromolecular pharmaceutical agent that hardly passes through gastrointestinal tracts to tissues or cells.

U.S. Pat. No. 7,780,982B1 has disclosed a hyaluronic acid derivative for improving the biodegradability thereof and the biodegradability of the micelle formed therewith, which has C₂˜C₁₆ hydrocarbons binding to the carboxyl group (—OH) of the hyaluronic acid and forms a urethane group.

However, studies have shown that, although drug-encapsulated micelles enter into cancer cells though endocytosis, they might be expelled by the cancer cell through exocytosis before the drug is released to kill the cancer cells. Thus, there is a demand for an improvement of the hyaluronic acid derivative and the micelle formed therewith.

### SUMMARY

The disclosure provides a hyaluronic acid derivative which comprises at least one repeating unit as shown in the following formula (I):

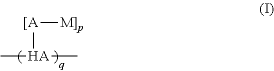

In the formula,

HA represents a unit comprising N-acetyl-D-glucosamine and D-glucuronic acid, q represents an integer of 2 to 10,000;

A represents a biocleavable linkage comprising at least one group of hydrazone, acetal, ketal and imine;

M represents at least one of a hydrophobic fragment, a hydrophilic fragment, and an amphiphilic fragment, and

p represents the number of [A-M] directly grafted onto each HA unit, p represents an integer of 0 to 4, and the p of each HA unit is not 0 at the same time;

wherein the hyaluronic acid derivative is biodegradable and pH-sensitive, and linkages of the hyaluronic acid derivative are broken in acidic conditions.

The disclosure also provides a micelle formed by the hyaluronic acid derivative described above in a hydrophilic medium.

The disclosure furthermore provides a drug delivery system which comprises a carrier and a bioactive ingredient encapsulated with the carrier, wherein the carrier consists of the hyaluronic acid derivative described above.

In addition, the disclosure provides a flavor enhancer for encapsulating a bioactive material to mask the flavor thereof, in which the flavor enhancer consists of the hyaluronic acid derivative described above.

### BRIEF DESCRIPTION OF THE DRAWINGS

FIG. 1 shows a chemical formula of HA-g-_{Hz}PCL in one example of the disclosure, in which HA represents a unit of hyaluronic acid, g represents grafting of chemical bonds, HZ represents a group of hydrazine and PCL represents a chain fragment of polycaprolactone;

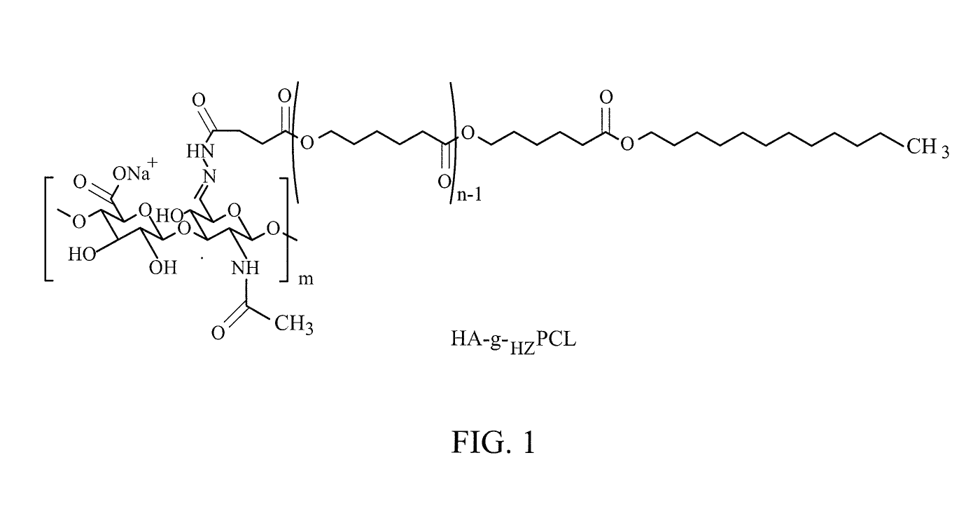

FIG. 2 shows a chemical formula of HA-g-(_{HZ}PCL-PEG) in one example of the disclosure, in which HA represents a unit of hyaluronic acid, g represents grafting of chemical bonds, HZ represents a group of hydrazone, PCL represents a chain fragment of polycaprolactone and PEG represents a chain fragment of polyethylene glycol;

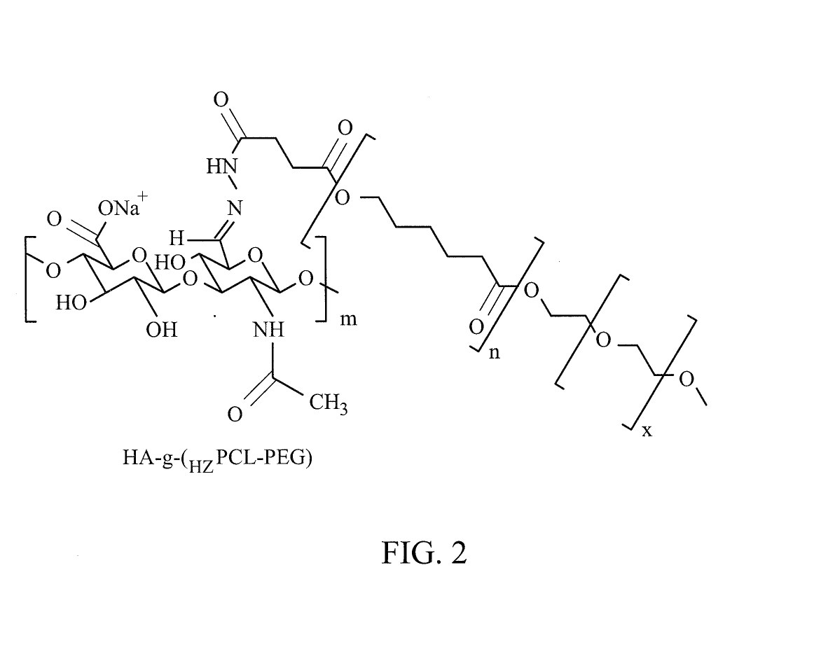

FIG. 3 shows ¹H-NMR spectra of HA-COONa and HA-TBA in one example of the disclosure;

FIG. 4 shows a ¹H-NMR spectrum of HA-TBA-CHO in one example of the disclosure;

FIG. 5 shows a FT-IR spectrum of PCL-hydrazide in one example of the disclosure;

FIG. 6 shows a ¹H-NMR spectrum of HA-g-(_{Hz}PCL) in one example of the disclosure, in which HA represents a unit of hyaluronic acid, g represents a chemical grafting, HZ represents a hydrazone linkage and PCL represents a chain fragment of polycaprolactone;

FIG. 7 shows a ¹H-NMR spectrum of HA-g-(_{HZ}PCL-PEG) in one example of the disclosure, in which HA represents a unit of hyaluronic acid, g represents a chemical grafting, HZ represents a hydrazone linkage, PCL represents a chain fragment of polycaprolactone and PEG represents a chain fragment of polyethylene glycol;

FIG. 8 shows the drug release curve of the micelle in one example of the disclosure; and

FIG. 9 shows the drug release curve of the micelle in one example of the disclosure.

### DETAILED DESCRIPTION

The disclosure, in one example, provides improved pH-sensitive materials for drug release control, which adopts biocompatible hyaluronic acids as the basis and uses biocleavable linkages connecting hydrophobic fragments, hydrophilic fragments, amphiphilic fragments or a combination thereof to form hyaluronic acid derivatives having repeat units as shown in the formula (I).

In the repeat unit shown in the following formula (I):

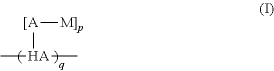

HA represents a hyaluronic acid unit comprising N-acetyl-D-glucosamine and D-glucuronic acid, and q represents the number of hyaluronic acid units. The q may be an integer of 2 to 10,000 in one example and may be an integer of 10 to 5,000 in another example, but it is not limited thereto. In the formula, A represents a biocleavable linkage, forming a covalent connection with at least one hydroxyl group (—OH) of the N-acetyl-D-glucosamine and the D-glucuronic acid. In the formula, M represents at least one of a hydrophobic fragment, a hydrophilic fragment and an amphiphilic fragment. The p represents the number of [A-M] directly grafted onto each HA unit, in which the biocleavable linkage binds to the hydroxyl group (—OH) of the N-acetyl-D-glucosamine and/or the D-glucuronic acid. Since the N-acetyl-D-glucosamine and the D-glucuronic acid in a hyaluronic acid contain 4 hydroxyl groups, p represents an integer of 0 to 4 and the p in each HA unit is not 0 at the same time. The disclosure does not specifically limit the linkage position of the biocleavable linkage to the hydroxyl group. However, for ease of understanding, the following description takes one biocleavable linkage (p=1) as an example.

The term “biocleavable linkage” in the disclosure refers to a linkage that is broken in acidic conditions. The biocleavable linkage covalently binds to the hydroxyl group (—OH) of the N-acetyl-D-glucosamine and/or the D-glucuronic acid, comprising hydrazone, acetal, ketal and/or imine. The “acidic condition” in the disclosure refers to a condition having a pH value less than 7. In one example, the pH value is 6.9˜1.0, and, in another example, the pH value is 6.5˜3.0, like the condition of cell organelles or cancer tissues.

The “M” in the disclosure refers to a hydrophobic fragment, a hydrophilic fragment, and/or an amphiphilic fragment. The molecular weight of M is not specifically limited, and may be 100 to 50,000 Da in one example, 300 to 30,000 Da in another example and 500 to 20,000 Da in one another example.

The “hydrophobic fragment” in the disclosure refers to a chain fragment consisting of bioabsorbable polymers as repeat units, such as caprolactone, butyrolactone, D-lactide, L-lactide, D-lactic acid, L-lactic acid, glycolide, glycolic acid, hydroxyl hexonoic acid, hydroxyl butyric acid, valerolactone, hydroxyl valeric acid, malic acid, a copolymer thereof or a combination thereof. The bioabsorbable polymer may also comprise one or more biocleavable linkages, such as hydrazone, acetal, ketal and imine.

The “hydrophilic fragment” in the disclosure refers to a hydrophilic chain fragment without a specific limitation. The hydrophilic fragment comprises polyethylene glycol (PEG), polyethylene oxide (PEO), polyvinylpyrrolidone (PVP), polyacrylic acid (PAA), polymethacrylic acid (PMA), or a combination thereof.

The “amphiphilic fragment” in the disclosure refers to an amphiphilic chain fragment comprising hydrophilic regions and hydrophobic regions. The hydrophilic region comprises repeat units comprising ethylene glycol, ethylene oxide, vinylpyrrolidone, acrylic acid, methacrylic acid, a copolymer thereof or a combination thereof. The hydrophobic region comprises repeat units comprising caprolactone, butyrolactone, D-lactide, L-lactide, D-lactic acid, L-lactic acid, glycolide, glycolic acid, hydroxyl hexonoic acid, hydroxyl butyric acid, valerolactone, hydroxy valeric acid, malic acid, a copolymer thereof or a combination thereof. The amphiphilic fragment may also comprise one or more biocleavable linkages, such as hydrazone, acetal, ketal and/or imine.

In another example, the A and M are the same as described above, and the HA may further bind to a hydrophilic fragment through a non-biocleavable linkage or a linkage that is not easily or quickly broken. The “non-biocleavable linkage” refers to the linkage which is not broken under acidic conditions, such as urethane linkage. The “linkage that is not easily or quickly broken” refers to a linkage that is not quickly broken in 24 hours under acidic conditions, such as ester linkage. The “hydrophilic fragment” bound through the non-biocleavable linkage or the linkage that is not easily or quickly broken may comprise polyethylene glycol (PEG), polyethylene oxide (PEO), polyvinylpyrrolidone (PVP), polyacrylic acid (PAA), polymethacrylic acid (PMA), or a combination thereof. The HA is bound to the hydrophilic fragment through the non-biocleavable linkage or the linkage that is not easily or quickly broken, grafted onto the hydroxyl group of HA at one end and bound to the hydrophilic fragment at the other end. The binding of the HA and the hydrophilic fragment can increase the solubility of the hyaluronic acid derivative in aqueous media and reduce the risk of micelles formed by the hyaluronic acid derivative being identified by the immune system in the circulation and thus increase the circulation period of the micelles in the blood.

In one example, the hyaluronic acid derivative may bind to a bio-identifiable protein molecule with an isolated chain fragment. The “isolated chain fragment” refers to a chain fragment binding to the HA at a position different to the binding of the biocleavable linkage and non-biocleavable linkage. For example, the isolated chain fragment can be carbon chains or carbon-oxygen chains which may have a carbon number of 1 to 1000. The isolated chain fragment may have a molecular weight of 100 to 50,000 Da. In one example, the molecular weight of the isolated chain fragment may be 300 to 30,000 Da. In another example, the molecular weight of the isolated chain fragment may be 500 to 20,000 Da. The “bio-identifiable protein molecule” may be an antibody or ligand without specific limitation. In one example, the protein molecule may be an antibody or ligand that has the ability to specifically identify specific species of cancer cells.

According to the disclosure, because the hydroxyl group is modified with a hydrophobic fragment or amphiphilic fragment, the hyaluronic acid derivative can self-assemble and form a nano-scaled micelle in a hydrophilic medium when the concentration is higher than the critical micelle concentration (CMC). The “hydrophilic medium” in the disclosure refers to a hydrophilic solvent, such as water, saline, blood, sera, ethanol or the like, and it is not limited thereto. The “critical micelle concentration” (CMC) in the disclosure refers to the concentration at which the hyaluronic acid derivatives form a micelle in a hydrophilic medium. The critical micelle concentration may be 10 to 0.0001% by weight based on the weight of the hydrophilic medium in one example.

According to the disclosure, the hyaluronic acid derivative may be mixed and vortexed with a bioactive ingredient to form a micelle encapsulating the bioactive ingredient. The bioactive ingredient may comprise drugs, vitamins, nutrients, or the like, in particular chemotherapeutic drugs like rhodamine, doxorubicin and paclitaxel. In one example of the disclosure, the ratio of the bioactive ingredient and the hyaluronic acid derivative which forms micelles are 1:1 to 1:20 by weight.

The “pH-sensitive” refers to a phenomenon that a chemical is hydrolyzed under acidic conditions. The hyaluronic acid derivative according to the disclosure shows pH-sensitive because the hyaluronic acid derivative has the biocleavable linkage which is going to break under acidic conditions.

In addition, when the micelles encapsulating bioactive ingredients enter animal blood, the micelles infiltrate and accumulate at the larger intercellular spacers between cells or tissues, especially cancer tissues, due to the enhanced permeation and retention effect (EPR effect). Since cancer tissues are mostly acidic, the biocleavable linkage of the hyaluronic acid derivative is going to quickly hydrolyzed and broken. The carried bioactive ingredient is therefore released and kills the cancer cells. On the other side, the micelle according to the disclosure may enter animal cells through endocytosis. The biocleavable linkage of the hyaluronic acid derivative is going to be quickly hydrolyzed and broken at the acidic intracellular condition, which enhances the quick release of the carried bioactive ingredient. In spite of the therapeutic effects, the fast acidic-hydrolysis and drug release can avoid micelles being expelled through exocytosis. In addition, in one example, the hyaluronic acid derivative binds to a bio-identifiable protein molecules, the micelle formed by the hyaluronic acid derivative can target specific cells, such as cancer cells, and deliver the carried bioactive ingredient specifically to the cells.

The disclosure also provides a flavor enhancer for masking flavors of drugs, nutrients or bioactive ingredients which have strong or stinky smells. The flavor enhancer is formed by the hyaluronic acid derivative described above which forms micelles to encapsulate the drugs or bioactive ingredients. Being flavor enhancers, the drugs, nutrients or bioactive ingredients encapsulated are not specifically limited. When oral administration or administration through an enteral route, the acidic condition of gastrointestinal tracts will enhance the micelle becoming hydrolyzed and degraded so that the encapsulated drugs or bioactive ingredients can be released. Therefore, the flavor enhancer is used appropriately for encapsulating nutrients which are administered orally or enterally.

In the following, one embodiment of production of the hyaluronic acid derivatives is described. However, the embodiment does not limit the invention.

For production of the hyaluronic acid derivative, first, hyaluronic acid and tetrabutylammonium hydroxide (TBAOH) is mixed by using ion absorption to produce hyaluronic acid-tetrabutyl ammonium (HA-TBA). The reaction is shown in the following.

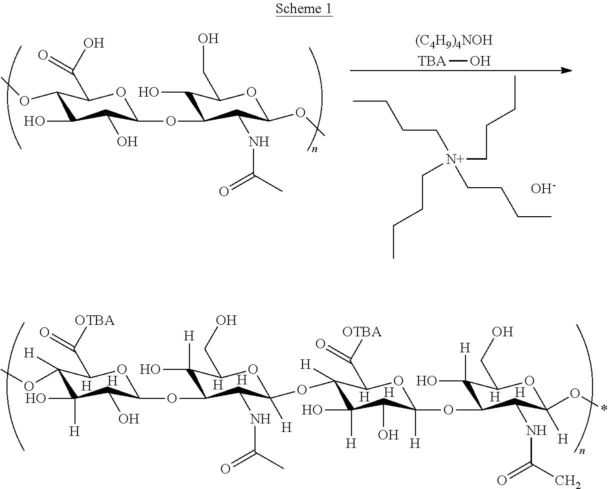

Subsequently, the hydroxyl group of the hyaluronic acid derivative is modified to pH-sensitive linkage. An oxidant, 2,2,6,6-tetramethyl-piperidin-1-oxyl (TEMPO), which selectively oxidizes primary alcohols to aldehydes, is adopted. The HA-TBA is reacted with TEMPO and [bis(acetoxy)iodo]benzene (BAIB), resulting in the hydroxyl group of the HA-TBA being oxidized. Thus, an intermediate product HA-TBA-CHO is obtained as follows.

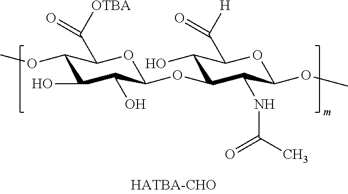

Thereafter, a hydrophobic chain fragment with hydrazide at the end is produced. The hydrophobic chain fragment is reacted with the aldehyde group of the HA-TBA-CHO to form pH-sensitive hydrazone. First, 1-dodecanol as an initiator is reacted with ε-caprolactone through ring-opening polymerization in the presence of stannous octoate as a catalyst under high temperature. A hydrophobic polymer, poly (ε-caprolactone) (PCL), is therefore obtained. Subsequently, a two-step modification of the end functional group is performed. At the first step, PCL-OH is deprotonized with triethylamine. The deprotonized PCL-OH is then reacted with succinic anhydride for ring-opening reaction in the present of 4-dimethylaminopyridine as catalysts. A PCL with modified carboxyl group at the end (PCL-COOH) is obtained. In the second step, the PCL-COOH is deprotonized with N-methylmorpholine (NMM) and then reacted with isobutylchloroformate (IBCF) for addition-elimination reaction. An intermediate product, PCL-anhydride, which has higher reactivity, is obtained. The intermediate product, PCL-anhydride, is then reacted with 1M hydrazine tetrahydrofurun solution. A hydrophobic PCL-hydrazide prepolymer with a modified hydrazide group at the end is obtained as follows.

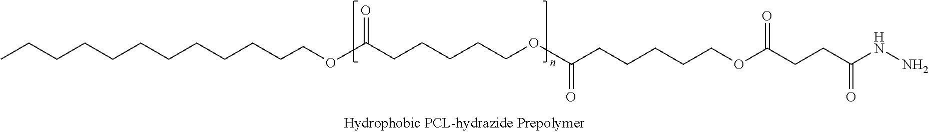

In one example, the PCL-hydrazide prepolymer is connected with a hydrophilic fragment, such as PEG, at the end of PCL to form an amphiphilic fragment as follows.

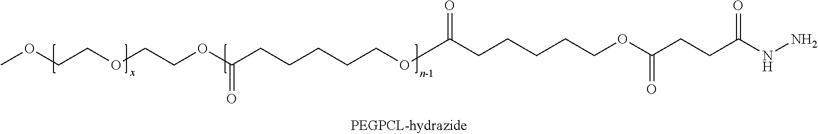

The PCL-hydrazide or the PEG-PCL-hydrazide prepolymer is then mixed with the HATBA-CHO prepolymer under specific conditions to obtain the desired grafting ratios, making the PCL-hydrazide or the PEG-PCL-hydrazide prepolymer form a hydrazone linkage. Finally, a pH-sensitive, biodegradable hyaluronic acid derivative, HA-g(Hz-PCL) copolymer or HA-g(Hz-PCL-PEG) copolymer is obtained as follows.

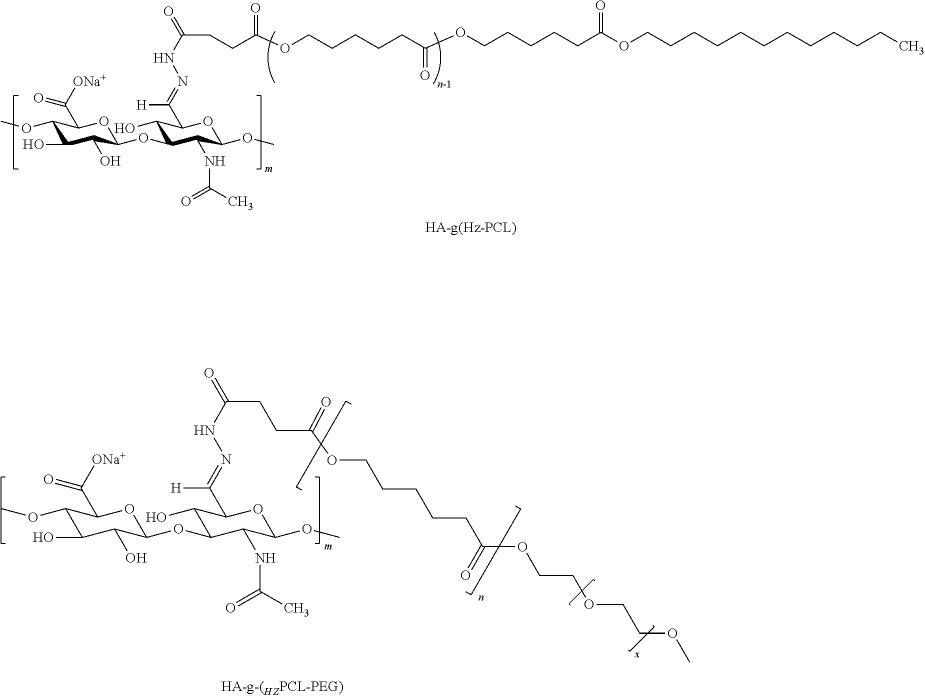

The following examples are used for describing the invention in detail; however, they are not used to limit the scope of the invention.

### Example 1

### Production of Hyaluronic Acid-Tetrabutyl Ammonium Hydroxide (HA-TBA)

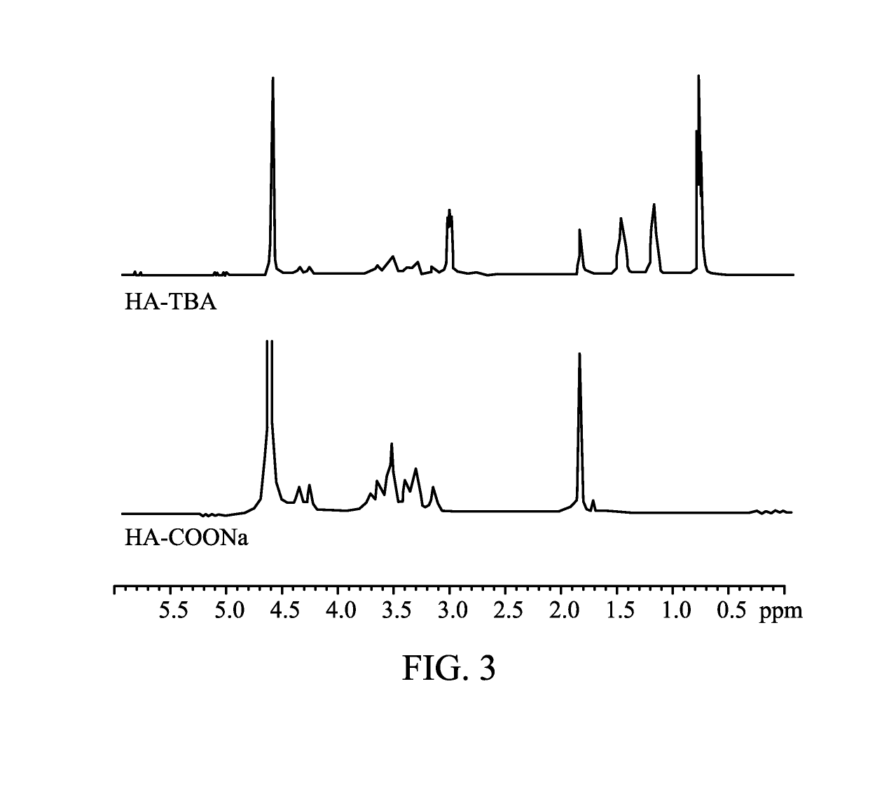

500 mL of hydrogen ion exchange resin (ROHM HAAS, for food application) was added into a chromatographic column and washed with double deionized water. Subsequently, 6 g of hyaluronic acid sodium salt powder (HA-COONa) (Mw=16,000) was added to 600 mL double deionized water to formulate 1% aqueous solution. The HA-COONa aqueous solution was then added to the hydrogen ion exchange resin column for the exchange of sodium ions and hydrogen ions. An intermediate product, carboxyl hyaluronic acid (HA-COOH), was obtained. The HA-COOH was then mixed with an equal molar volume of tetrabutylammonium hydroxide (Fluka) (40%, 9.8 mL) and stirred at room temperature for 16 hours. After being freeze-dried, 9.2 g white sheets, hyaluronic acid-tetrabutyl ammonium hydroxide (HA-TBA), was obtained (yields 99%). The HA-TBA was subsequently analyzed by NMR spectroscopy. The ¹H NMR spectrum is shown in FIG. 3.

### Example 2

### Production of Hyaluronic Acid-Tetrabutylammonium Hydroxide-Aldehyde (HA-TBA-CHO)

4 g of the HA-TBA obtained in Example 1 (6.44 mmol) was added to a 250 mL-two-neck bottle and dried under vacuum at room temperature for 2 hours. Then, 71.7 mL of anhydrous dimethylformamide was added to the bottle. After the HA-TBA was dissolved, 5.41 g of sodium carbonate (64.4 mmol) and 0.403 g of 2,2,6,6-tetramethyl-piperidin-1-oxyl (2.58 mmol) were added to the HA-TBA solution. The mixture was cooled to 0° C. and subsequently, 2.07 g of iodobenzene diacetate (6.44 mmol) was added thereto. The bottle was then returned to room temperature for a 6-hour reaction, and a crude product of hyaluronic acid-tetrabutylammonium hydroxide-aldehyde (HA-TBA-CHO) was obtained. The crude product was then vortexed in the vacuum under reduced pressure until the solution become orange-red, thick, and the volume was reduced to 20 mL. Then, 1000 mL of ethyl acetate was slowly dripped into the solution and precipitates were appeared. After being centrifuged, white solids of the crude product HA-TBA-CHO were obtained. The crude product HA-TBA-CHO was then mixed with 20 mL of ethanol. After the HA-TBA-CHO was dissolved, a mixed solution of ether and tetrahydrofuran (ether:tetrahydrofuran=2:1) was slowly dripped into the HA-TBA-CHO solution and white solid precipitates appeared. The solution was then centrifuged to remove the solvents. After concentrated under reduced pressure and dried in vacuum at room temperature for one day, white solids of the product HA-TBA-CHO 3.05 g were obtained. The transition rate of HA-TBA-CHO from HA-TBA with stoichiometric adjustment is listed in Table 1.

TABLE 1

| Equivalent Ratio | | | | | |
|---|---|---|---|---|---|
| HA-TBA | TEMPO | BAIB | Solvents | S.C.(%) | Conversion(%) |
| 1 | 0.2 | 3 | DMAc | 10 | 14 |
| 1 | 0.2 | 3 | DMF | 10 | 50 |
| 1 | 0.4 | — | DMF | 40 | 48 |
| 1 | 0.4 | 3 | DMF | 40 | 48 |
| 1 | 0.4 | 3 | DMF | 12 | 84 |
| 1 | 0.4 | 3 | DMF | 12 | 84 |
| 1 | 0.4 | 1 | DMF | 12 | 51 |
| 1 | 0.2 | 1 | DMF | 12 | 31 |

*HA-TBA: Hyaluronic acid-tetrabutylammonium;
TEMPO: 2,2,6,6-tetramethyl-piperidin-1-oxyl;
BAIB: [bis(acetoxy)iodo]benzene;
S.C.: solid content.

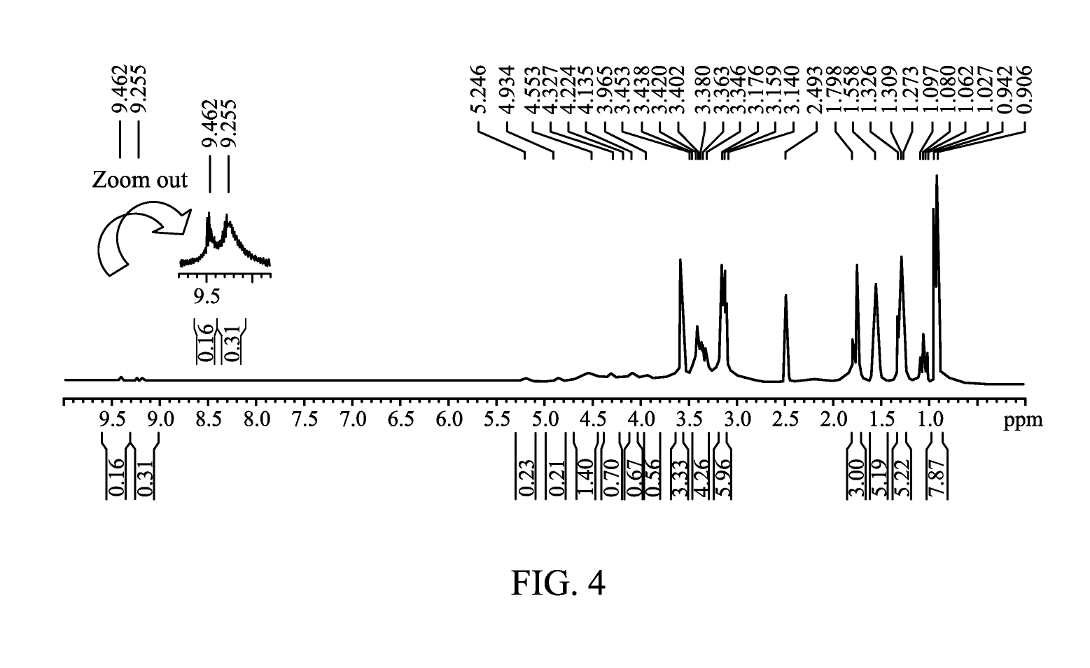

The NMR spectrum of HA-TBA-CHO is shown in FIG. 4. The aldehyde signal of HA-TBA-CHO is shown at δ 9.46 ppm and δ 9.26 ppm. The conversion is calculated as compared to the signal of the hydrogen atom at position 12 of the HA.

### Example 3

### Production of Carboxy-Polycaprolactone (PCL-COOH)

1-dodecanol was put into a glass reactor and added with stannous octoate (Sn(Oct)₂). The reactor was then heated to 130° C. Subsequently, ε-caprolactone monomers were slowly added into the reactor for polymerization. After the polymerization was finished, the reactor was cooled to room temperature and added with an equal volume of dichloromethane. After the solution was dissolved, diethyl ether was added to the solution at 4° C. and the solution was stand at −20° C. for 1 hour. White precipitates were appeared and then collected with filtration and vacuum-dried at room temperature for 24 hours. White solids, polycaprolactone monool (PCL-OH), were then obtained. The PCL-OH products with molecular weights are listed in Table 2 with stoichiometric adjustment.

TABLE 2

| PCL-OH | C/D^{a} | Sn(Oct)₂ | Mw | Mn | PDI |
|---|---|---|---|---|---|
| Sample 1 | 20.18/1 | 0.5 mol%^{b} | 4281 | 3071 | 1.39 |
| Sample 2 | 15.00/1 | 0.5 mol%^{b} | 4815 | 3521 | 1.37 |
| Sample 3 | 10.53/1 | 0.5 mol%^{b} | 4108 | 2933 | 1.40 |
| Sample 4 | 5.26/1 | 0.5 mol%^{b} | 1387 | 1172 | 1.19 |
| Sample 5 | 10.53/1 | 0.5 mol%^{b} | 2573 | 2093 | 1.23 |
| Sample 6 | 5.26/1 | 1 mol%^{c} | 1267 | 1079 | 1.17 |
| Sample 7 | 5.26/1 | 0.5 mol%^{c} | 1152 | 1005 | 1.15 |
| Sample 8 | 43.81/1 | 0.5 mol%^{c} | 5154 | 3979 | 1.30 |
| Sample 9 | 20.18/1 | 0.5 mol%^{c} | 2706 | 2203 | 1.23 |
| Sample 10 | 43.81/1 | 0.5 mol%^{c} | 3419 | 2808 | 1.22 |
| Sample 11 | 20.18/1 | 0.5 mol%^{c} | 2740 | 2199 | 1.25 |
| Sample 12 | 43.81/1 | 0.5 mol%^{c} | 7686 | 4696 | 1.63 |

^{a}C/D: the ratio of caprolactone to 1-dodecanol;
^{b}mol %: molar ratio compared to caprolactone;
^{c}mol %: molar ratio compared to 1-dodecanol;
PDI: distribution of molecular weights.

The obtained PCL-OH 6 g (Mw=4281, 1.37 mmol) was then added to a 100 mL-two-neck bottle and vacuum dried. Then 15.5 mL of anhydrous tetrahydrofuran was added into the bottle under nitrogen atmosphere. The bottle was then heated to 60° C. After the solution was dissolved, 1.2 mL of triethylamine was added into the bottle. Meanwhile, 0.121 g of 4-dimethylaminopyridine (DMAP) (0.992 mmol) and 0.844 g of succinic anhydride (8.43 mmol) were dissolved in 16 mL anhydrous tetrahydrofuran. Subsequently, the PCL-OH solution obtained above was slowly dripped into the mixture of DMAP and succinic anhydride and reacted at room temperature for 48 hours. After being concentrated under reduced pressure to remove the solvent tetrahydrofuran, crude products of PCL-COOH were obtained.

Meanwhile, 600 mL of a mixed solution of ether and petroleum ether (1:1, v/v) was prepared. The crude product PCL-COOH obtained above was slowly added to the mixed solution of ether and petroleum ether. After white precipitates appeared, the solution was cooled to −20° C. and stand for 2 hours. The white precipitates were collected with vacuum filtration. After being vacuum dried at room temperature, 4.6 g of PCL-COOH was obtained.

### Example 4

### Production of Polycaprolactone-Hydrazide (PCL-Hydrazide)

12 g of the PCL-COOH obtained in Example 3 (Mw=3576, 3.36 mmol) was added to a 100 mL-two-neck bottle and vacuumed. 54 mL of anhydrous tetrahydrofuran was then added to the bottle in the nitrogen atmosphere. The bottle was heated to 60° C. for complete dissolution and then cooled to room temperature. Subsequently, 1.9 mL of N-methylmorpholine (NMM) (16.78 mmol) was slowly dripped into the bottle. The bottle was then cooled to 0° C. Then, 2.2 mL of isobutyl chloroformamide (IBCF) (16.78 mmol) was added to the bottle. After stirring for 30 minutes at room temperature, an intermediate product, PCL-anhydride, in a clear solution with white salts was obtained.

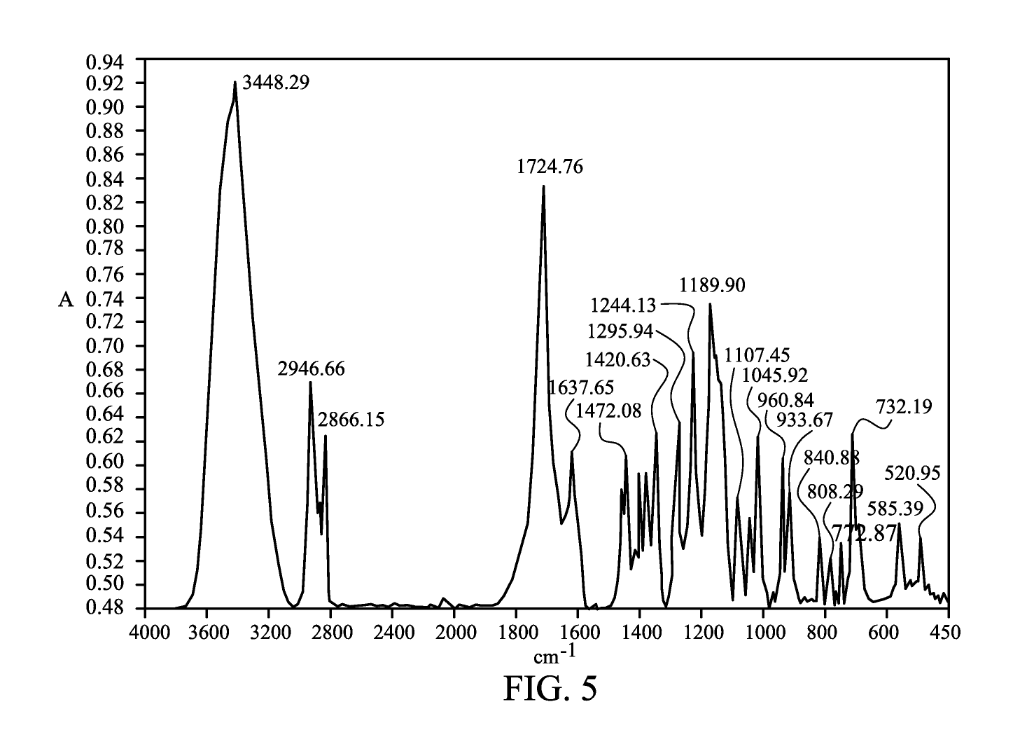

The obtained clear solution containing the intermediate product PCL-anhydride was slowly dripped to a 150 mL-two-neck bottle which contained 33.6 mL of 1M hydrazine in tetrahydrofuran (THF) (33.6 mmol). The bottle was heated to 40° C. for a 16-hour reaction. The reactants were concentrated under reduced pressure, removing the solvent THF. Crude products, polycaprolactone-hydrazide (PCL-hydrazide), were obtained. The obtained crude product PCL-hydrazide was then slowly dripped into a mixture of ether and petroleum ether (1:1, v/v) and white precipitates were appeared. The solution was then cooled to −20° C. and stand for 2 hours. The white precipitates were then collected with filtration and vacuum-dried at room temperature. The product, PCL-hydrazide, was obtained (7.44 g). The product PCL-hydrazide was then analyzed. The FT-IR spectrum of the product PCL-hydrazide is shown in FIG. 5, in which the absorption peak of the amide group of the PCL-hydrazide is at 1637 cm⁻¹.

### Example 5

### Production of Polyethylene Glycol-Polycaprolactone-Hydrazide (PEG-PCL-Hydrazide)

A 250 mL-glass column was set as the reactor, heated to 100° C. and supplied with nitrogen air for 30 minutes. Thereafter, 120 g of methoxypolyethylene glycol (mPEG) (molecular weight 5000 g/mole) and 48 g of ε-caprolactone were added to the reactor. The reactor was heated slowly until the reactants all dissolved. When the temperature reached 100° C., a catalyst, stannous octoate (Sn(Oct)₂), 0.67 ml was added to the reactor and reacted at 130° C. for 24 hours. The obtained product was then dissolved in dichloromethane and precipitated with ether. The reactants were vacuum-dried at 25° C. for 24 hours. White powders, polyethylene glycol-polycaprolactone-hydroxide (PEG-PCL-OH), were obtained. The PEG-PCL-OH with different chain lengths by using various stoichiometric ratios are shown in Table 3.

TABLE 3

| | mPEG | Molar Ratio | | | |
|---|---|---|---|---|---|
| PEG-PCL-OH | (Mw) | (PEG:PCL) | Mw | Mn | PDI |
| Sample 13 | 550 | 1:8 | 3477 | 2874 | 1.22 |
| Sample 14 | 550 | 1:10 | 3273 | 2597 | 1.26 |
| Sample 15 | 550 | 1:16 | 5299 | 4290 | 1.24 |
| Sample 16 | 1900 | 1:7 | 2781 | 2644 | 1.05 |
| Sample 17 | 1900 | 1:18 | 4928 | 3883 | 1.27 |
| Sample 18 | 1900 | 1:35 | 7973 | 5314 | 1.50 |
| Sample 19 | 5000 | 1:25 | 6350 | 5533 | 1.14 |

*PDI: distribution of molecular weights.

The obtained PEG-PCL-OH, Sample 17, 12 g (Mw=4928, 2.435 mmol) was added to a 100 mL-two-neck bottle. The bottle was dried in vacuum to remove water. Then, 14.1 mL of anhydrous tetrahydrofuran was added to the bottle in the nitrogen atmosphere. The bottle was heated to 60° C. After the PEG-PCL-OH was uniformly dissolved, 1.1 mL of triethylamine (7.305 mmole) was added to the bottle. Meanwhile, 0.119 g of 4-dimethylaminopyridine (DMAP) (0.974 mmol) and 0.828 g of succinic anhydride (8.28 mmol) were completely dissolved in 25 ml anhydrous tetrahydrofuran. Thereafter, the PEG-PCL-OH solution above was slowly dripped into the mixture of DMAP and succinic anhydride. The reaction was performed at room temperature for 48 hours and then concentrated at reduced pressure to remove the solvent tetrahydrofuran. Crude products, PEG-PCL-COOH (PEG₁₉₀₀-PCL₃₀₀₀-COOH), were thus obtained. The obtained crude product PEG₁₉₀₀-PCL₃₀₀₀-COOH was then slowly dripped into a mixture of ether and petroleum ether (1:1, v/v) and white solids were precipitated. The solution was then cooled to −20° C. and stand for 2 hours. The white precipitates were then collected with filtration and vacuum-dried at room temperature. The product, PEG-PCL-COOH (PEG₁₉₀₀-PCL₃₀₀₀-COOH), was thus obtained (11.06 g). The PEG-PCL-COOH products with various molecular weights by using different stoichiometric ratio are listed in Table 4.

TABLE 4

| PEG-PCL- | PEG | PCL | mPEG-PCL-OH | SA | MAP | Et₃N | HF | Yield |
|---|---|---|---|---|---|---|---|---|
| COOH | (Mw) | (Mw) | (g) | (g) | (g) | (ml) | (ml) | (%) |
| Sample 20 | 550 | 3000 | 5 | 0.489 | 0.07 | 0.6 | 15.1 | 81.2 |
| Sample 21 | 550 | 5000 | 20 | 1.284 | 0.184 | 1.6 | 60.4 | 59.0 |
| Sample 22 | 1900 | 900 | 12 | 1.458 | 0.209 | 1.8 | 42.0 | 84.1 |
| Sample 23 | 1900 | 3000 | 12 | 0.828 | 0.119 | 1.1 | 39.1 | 92.1 |
| Sample 24 | 1900 | 6000 | 12 | 0.512 | 0.073 | 0.63 | 38.0 | 96.3 |
| Sample 25 | 5000 | 1350 | 12 | 0.643 | 0.092 | 0.79 | 38.4 | 93.3 |

SA: succinic anhydride; DMAP: 4-dimethylamino pyridine, Et₃N: triethylamine; THF: tetrahydrofuran.

The obtained product PEG₁₉₀₀-PCL₃₀₀₀-COOH, Sample 23 (Mw=4928, 2.03 mmol), 10 g was added to a 100 mL-two-neck bottle. The bottle was vacuum-dried to remove water. Then, 45 mL of anhydrous tetrahydrofuran was added to the bottle in the nitrogen atmosphere. The bottle was heated to 60° C. to make the product uniformly dissolved and then cooled to room temperature. Subsequently, 1.1 mL of N-methylmorpholine (NMM) (10.15 mmol) was added to the bottle. Until the bottle temperature reached 0° C., 2.2 mL of isobutyl chloroformamide (IBCF) (10.15 mmol) was added to the bottle. After stirring at room temperature for 30 minutes, an intermediate product, PEG₁₉₀₀-PCL₃₀₀₀-anhydride, in a clear solution was obtained with white salts. The clear solution with the intermediate product PCL-anhydride was slowly dripped to a 150 mL-two-neck bottle which contained 20.3 mL of 1M hydrazine in THF (20.03 mmol). The bottle was heated to 40° C. for a 16-hour reaction. The reactants were concentrated under reduced pressure to remove the solvent THF. Crude products, PEG₁₉₀₀-PCL₃₀₀₀-hydrazide, were obtained. The obtained crude product PEG₁₉₀₀-PCL₃₀₀₀-hydrazide was then slowly dripped into a mixture of ether and petroleum ether (1:1, v/v) and white solids were precipitated. The solution was then cooled to −20° C. and stand for 2 hours. The white precipitates were then collected with filtration and dried at room temperature in vacuum. White powders, PEG₁₉₀₀-PCL₃₀₀₀-hydrazide, were thus obtained (8.76 g). The PEG-PCL-hydrazide products with various PEG-PCL-COOH chain lengths were obtained using different stoichiometric ratios. The results are shown in Table 5.

TABLE 5

| | | | mPEG-PCL- | | | 1M NH₂NH₂ | | |
|---|---|---|---|---|---|---|---|---|
| PEG-PCL- | mPEG | PCL | COOH | IBCF | NMM | in THF | THF | Yield |
| hydrazide | (Mw) | (Mw) | (g) | (ml) | (ml) | (ml) | (ml) | (%) |
| Sample 26 | 550 | 3000 | 10 | 1.9 | 1.6 | 28.8 | 45.1 | 83.6 |
| Sample 27 | 550 | 5000 | 10 | 1.2 | 1.1 | 18.9 | 45.1 | 90.8 |
| Sample 28 | 1900 | 900 | 10 | 2.3 | 2.0 | 35.7 | 45.0 | 51.2 |
| Sample 29 | 1900 | 3000 | 10 | 1.0 | 0.85 | 15.5 | 45.1 | 87.6 |
| Sample 30 | 1900 | 6000 | 10 | 0.8 | 0.7 | 12.5 | 45.0 | 90.8 |
| Sample 31 | 5000 | 1350 | 10 | 1.0 | 0.85 | 15.5 | 45.1 | 90.1 |

IBCF: isobutyl chloroformamide; NMM: N-methylmorpholine; NH₂NH₂ in THF: hydrazine in tetrahydrofuran; THF: tetrahydrofuran.

### Example 6

### Production of Hyaluronic acid-g-(hydrazone-polycaprolactone) [HA-g-(_{HZ}PCL)]

0.5 g of the HA-TBA-CHO (0.805 mmol) obtained in Example 2 was added to a 25 mL-two-neck bottle and completely dissolved with 5.6 mL of absolute ethanol. Meanwhile, 0.344 g of the PCL-hydrazide (0.0805 mmol) obtained in Example 4 was dissolved in 4 mL of absolute ethanol. The PCL-hydrazide in ethanol was slowly dripped into the HA-TBA-CHO ethanol solution above. The reaction was performed at 65° C. for 8 hours and then cooled down to room temperature. Crude products, HA-TBA_{16K}-g-(_{Hz}PCL) (graft ratio of PCL: 10%), were obtained.

Subsequently, the obtained crude product HA-TBA_{16K}-g-(_{HZP}CL) was added into a dialysis membrane (molecular weight cut-off (MWCO) 12,000˜14,000) and was purified at 16° C. The dialysis process comprised the first step in 500 mL of DMSO on Day 1, the second step in a saturated NaCl solution (pH=8) on Days 2 and 3, and the third step in double deionized water (pH=8) on Days 4 and 5. The purified product was then added into a sodium ion exchange resin for substitution of TBA. Then, a purified HA-g-(_{HZ}PCL) aqueous solution was obtained. After being freeze-dried, the final product HA-g-(_{HZ}RCL) (graft ratio of PCL: 10%) was obtained.

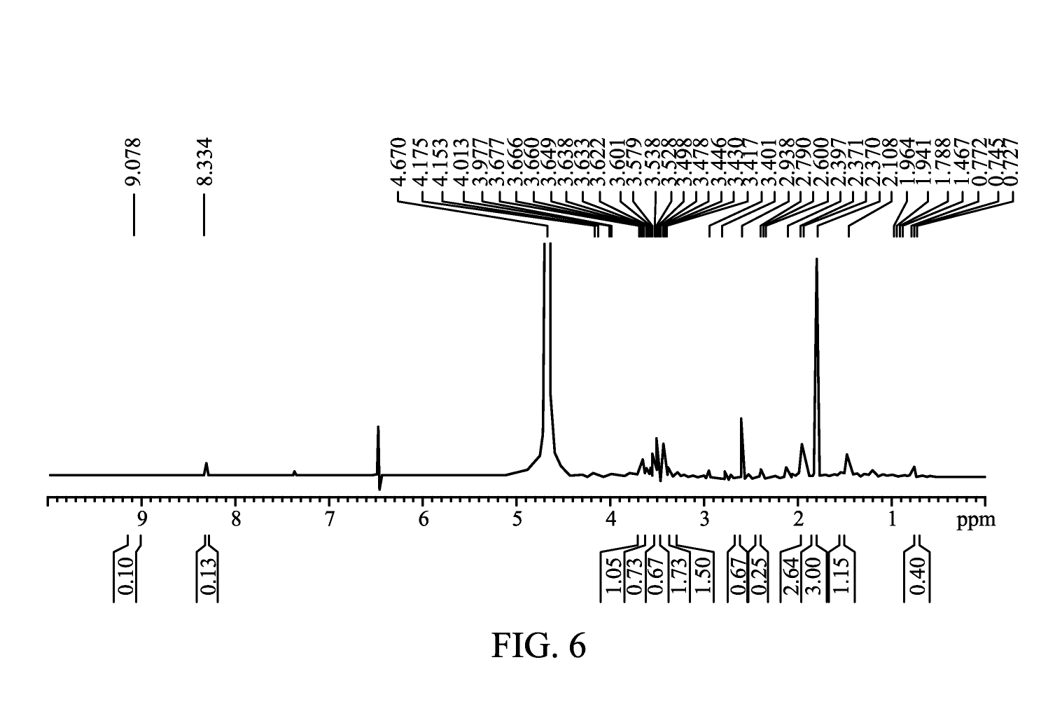

The final product HA-g-(_{HZ}PCL) was analyzed under ¹H-NMR spectroscopy. The resulting spectrum is shown in FIG. 6. The graft ratio of PCL is calculated by the proton signals of hydrazone linkage (δ 8.33 ppm, s) based on the proton signal at position 12 of PCL (δ 1.97 ppm, s).

### Example 7

### Production of Hyaluronic acid-g-(hydrazone-polycaprolactone-polyethylene glycol) [HA-g-(_{HZ}PCL-PEG)]

2 g of the HA-TBA-CHO (3.22 mmol) obtained in Example 2 was added to a 100 mL-two-neck bottle and completely dissolved with 20 mL of absolute ethanol. Meanwhile, the Sample 13 obtained in Example 5 (PEG₁₉₀₀-PCL₃₀₀₀-hydrazide) 1.06 g (0.39 mmol) was dissolved in 15 mL of absolute ethanol. The PEG₁₉₀₀-PCL₃₀₀₀-hydrazide in ethanol was slowly dripped into the HA-TBA-CHO ethanol solution above. The reaction was performed at 65° C. for 8 hours and then cooled down to room temperature. Crude products, HA-TBA_{16K}-g-(_{HZ}PCL₃₀₀₀-PEG₅₅₀) (graft ratio of PCL₃₀₀₀-PEG₅₅₀: 12%), were obtained.

The obtained crude product HA-TBA_{16K}-g-(_{HZ}PCL₃₀₀₀-PEG₅₅₀) was then added into a dialysis membrane (MWCO 12,000˜14,000) and was purified at 16° C. The dialysis process comprised the first step in 500 mL of DMSO on Day 1, the second step in saturated NaCl solution (pH=8) on Days 2 and 3, and the third step in double deionized water (pH=8) on Days 4 and 5. The purified product was then added into a sodium ion exchange resin for substitution of TBA. Then, a purified HA-g-(_{HZ}PCL₃₀₀₀-PEG₅₅₀) aqueous solution was obtained. After being freeze-dried, the final product, HA-g-(_{HZ}PCL₃₀₀₀-PEG₅₅₀) in the form of yellow solids (graft ratio of PCL₃₀₀₀-PEG₅₅₀: 12%), was obtained. The products HA₁₆ₖ-g-(_{Hz}PCL₃₀₀₀-PEG₅₅₀) obtained by using PEG₅₅₀-PCL₃₀₀₀-hydrazides in different chain lengths and stoichiometric ratios are shown in Table 6.

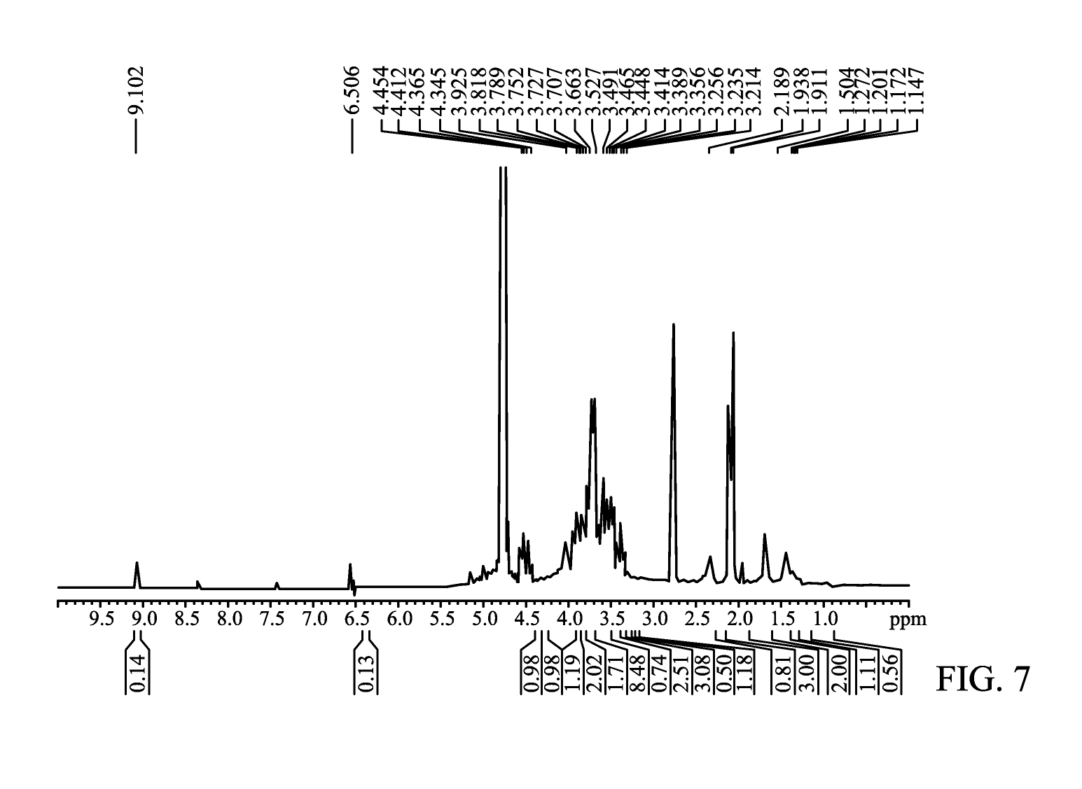

Meanwhile, the final product HA-g-(_{HZ}PCL₃₀₀₀-PEG₅₅₀) was analyzed under ¹H-NMR spectroscopy. The resulting spectrum is shown in FIG. 7. The graft ratio of PCL is calculated by the proton signals of hydrazone linkage h1 (δ 9.10 ppm, s) and h2 (δ 6.51 ppm, s) based on the proton signal at position 12 of PCL (δ 1.91 ppm, s).

TABLE 6

| | | PEG-PCL | | PEG-b-PCL- | | |
|---|---|---|---|---|---|---|
| HA-g-(_{Hz}PCL- | | Graft ratio | HATBA-CHO | hydrazine | EtOH | Yield |
| PEG) | PEG-PCL | (%) | (g) | (g) | (ml) | (%) |
| Sample 32 | PEG₅₅₀-PCL₃₀₀₀ | 5 | 1.5 | 0.42 | 22.0 | 23.7% |
| Sample 33 | PEG₅₅₀-PCL₃₀₀₀ | 6 | 2.0 | 0.531 | 29.0 | 68.60% |
| Sample 34 | PEG₅₅₀-PCL₃₀₀₀ | 12 | 2.0 | 1.063 | 35.0 | 62.00% |
| Sample 35 | PEG₅₅₀-PCL₃₀₀₀ | 12 | 5.0 | 2.657 | 87.3 | 53.70% |
| Sample 36 | PEG₅₅₀-PCL₃₀₀₀ | 20 | 2.0 | 1.44 | 43.0 | 41.50% |
| Sample 37 | PEG₁₉₀₀-PCL₃₀₀₀ | 20 | 3.0 | 4.76 | 88.5 | 77.80% |
| Sample 38 | PEG₁₉₀₀-PCL₃₀₀₀ | 6 | 2.0 | 0.952 | 33.7 | 69.80% |
| Sample 39 | PEG₁₉₀₀-PCL₃₀₀₀ | 12 | 2.0 | 1.904 | 44.5 | 43.10% |
| Sample 40 | PEG₁₉₀₀-PCL₃₀₀₀ | 12 | 5.0 | 4.76 | 111.3 | 80% |

### Example 8

### Production of Hyaluronic acid-g-(hydrazone-polycaprolactone-polyethylene glycol) [HA-g-(PCL-PEG)]

The PEG-PCL-OH, Sample 13 in Example 5, 1.87 g (0.54 mmol) was added to a two-neck bottle and azeotropic distillated with toluene at 65˜70° C. to remove water.

Subsequently, 4.8 mL of dimethyl sulfoxide (DMSO) was added to the bottle and the PEG-PCL-OH was dissolved therein. 6000 ppm of triethylene diamine (DABCO) and 3000 ppm of stannous octoate (Sn II) as catalysts were added to the bottle. Then, dicyclohexylmethane diisocyanate (H₁₂MDI, 0.23 mL, 0.48 mmol) was added to the bottle and reacted at 60° C. for 6 hours. A solution of prepolymer PEG-PCL-NCO was obtained.

Meanwhile, 3 g of the HA₁₆ₖ-TBA (4.84 mmol) obtained in Example 2 was added to a 50 mL-two-neck bottle. The bottle was added with 13 mL DMSO and heated to 60° C. After the solution was completely dissolved, 6000 ppm of triethylene diamine (DABCO) and 3000 ppm of stannous octoate (Sn II) was added as catalysts. Thereafter, the solution of prepolymer PEG-PCL-NCO obtained above was added to the bottle containing the HA-TBA prepared above. The reaction was under 60° C. for 16 hours. Crude products, HA-g-(PCL-PEG), were thus obtained (graft ratio of PCL-PEG: 10%).

The obtained crude product HA-g-(PCL-PEG) was added into a dialysis membrane (MWCO 12,000˜14,000) and was purified at 16° C. The dialysis process comprised the first step in 500 mL of DMSO on Day 1, the second step in saturated NaCl solution (pH=8) on Day 2 and 3, and the third step in double deionized water (pH=8) on Day 4 and 5. The purified product was then added into a sodium ion exchange resin for substitution of TBA. Then, a purified HA-g-(PCL-PEG) aqueous solution was obtained. After frozen and dried, the final product HA-g-(PCL-PEG) as yellow solids (graft ratio of PCL-PEG: 10%) was obtained. The products HA-g-(PCL-PEG) obtained by using PEG-PCLs in different chain lengths and stoichiometric ratio are shown in Table 7.

TABLE 7

| | | | | | | | | (II)HA-g-(PCL-PEG) | | | | |
|---|---|---|---|---|---|---|---|---|---|---|---|---|
| | | | (I)PEG-PCL-NCO | | | | | | PCL- | | | |
| | | PEG-PCL | | | Catalysts | | Solvents | | PEG- | Catalysts | | Solvents |
| HA-g- | | Graft ratio | PEG-PCL | H₁₂MDI | Sn(II) | DABCO | DMSO | HA-TBA | NCO | Sn(II) | DABCO | DMSO |
| (PCL-PEG) | PEG-PCL | (%) | g | ml | (ml) | (mg) | (ml) | g | g | (ml) | (mg) | (ml) |
| Sample 41 | PEG₅₅₀-PCL₃₀₀₀ | 10 | 1.87 | 0.12 | 5 | 12 | 6.8 | 3 | 1.81 | 11.5 | 29 | 15 |
| Sample 42 | PEG₅₀₀-PCL₃₀₀₀ | 20 | 3.74 | 0.25 | 10 | 24 | 13.6 | 3 | 3.62 | 15.9 | 40 | 17 |
| Sample 43 | PEG₁₉₀₀-PCL₃₀₀₀ | 6 | 1.06 | 0.05 | 3 | 7 | 3.8 | 2 | 1.00 | 7.2 | 18 | 13 |
| Sample 44 | PEG₁₉₀₀-PCL₃₀₀₀ | 20 | 3.53 | 0.17 | 9 | 22 | 12.8 | 2 | 3.35 | 12.8 | 32 | 15 |
| Sample 45 | PEG₁₉₀₀-PCL₃₀₀₀ | 10 | 2.14 | 0.06 | 5 | 13 | 7.8 | 1.5 | 1.99 | 8.4 | 21 | 15 |

### Example 9

### Analysis of Critical Micelle Concentration

The samples obtained above were separately formulated to 1 mg/ml aqueous solutions. The aqueous solutions were then aliquoted to obtain 15 concentrations until the concentration reached 6×10⁻⁵ mg/ml. Each aliquots were uniformly mixed with 5 p 1 of pyrene in acetone (1.8×10⁻⁴ M). The mixtures were left to stand in the dark for 16 hours and then the acetone was evaporated under vacuum. Subsequently, each aliquot was analyzed by a fluorescence spectrometer, emission was detected at wavelength 390 nm and an exciting wavelength of excitation spectrum was scanned from 270 to 360 nm. The wavelengths which showed the strongest absorption between 330-340 nm were recorded. According to the log of the concentration and the absorption on the fluorescence spectra, the point at which the absorption started changing indicated the critical micelle concentration. Table 8 shows the critical micelle concentration (CMC) of several micelle materials.

TABLE 8

| | Theoretic Graft Ratio/ | |
|---|---|---|
| | NMR Experimental | Critical Micelle |
| | Graft Ratio | Concentration |
| Micelle Materials | (%) | (CMC) |
| HA-g-(_{HZ}PCL₃₀₀₀-PEG₅₅₀) | 6/6 | 1.69 × 10⁻² |
| HA-g-(_{HZ}PCL₃₀₀₀-PEG₅₅₀) | 12/13 | 1.36 × 10⁻² |
| HA-g-(_{HZ}PCL₃₀₀₀-PEG₅₅₀) | 12/10 | 1.95 × 10⁻² |
| HA-g-(_{HZ}PCL₃₀₀₀-PEG₅₅₀) | 20/20 | 1.10 × 10⁻² |
| HA-g-(_{HZ}PCL₃₀₀₀-PEG₁₉₀₀) | 20/20 | 1.70 × 10⁻² |
| HA-g-(_{HZ}PCL₃₀₀₀-PEG₁₉₀₀) | 6/5 | 2.56 × 10⁻² |
| HA-g-(_{HZ}PCL₃₀₀₀-PEG₁₉₀₀) | 12/12 | 1.51 × 10⁻² |
| HA-g-(_{HZ}PCL₃₀₀₀-PEG₁₉₀₀) | 12/10 | 8.46 × 10⁻³ |
| HA-g-(_{HZ}PCL₆₀₀₀-PEG₁₉₀₀) | 4/2 | 7.70 × 10⁻³ |
| HA-g-(_{HZ}PCL₆₀₀₀-PEG₁₉₀₀) | 10/8 | 7.37 × 10⁻³ |
| HA-g-(_{HZ}PCL₁₃₅₀-PEG₅₀₀₀) | 50/50 | 1.50 × 10⁻³ |
| HA-g-(_{HZ}PCL₉₀₀-PEG₁₉₀₀) | 60/11 | 1.40 × 10⁻² |
| HA-g-(PCL₃₀₀₀-PEG₅₅₀) | 10/13 | 2.10 × 10⁻² |
| HA-g-(PCL₃₀₀₀-PEG₅₅₀) | 20/23 | 1.78 × 10⁻³ |
| HA-g-(PCL₃₀₀₀-PEG₁₉₀₀) | 6/7 | 3.70 × 10⁻³ |
| HA-g-(PCL₃₀₀₀-PEG₁₉₀₀) | 20/21 | 3.50 × 10⁻³ |
| HA-g-(PCL₆₀₀₀-PEG₁₉₀₀) | 10/17 | 5.40 × 10⁻³ |

### Example 10

### Particle Size Analyses

Each of the samples was weighted to 20 mg and added with 2 mL of DMSO for a 20-minute-vortex. Then, 1 mL double deionized water was added to the mixture and vortexed a further 20 minutes at room temperature until the mixture was completely dissolved. The obtained solutions were dialyzed with 1000 ml double deionized water in a dialysis membrane (MWCO 6000˜8000) for 24 hours. DMSO was removed. After the dialysis was finished, the solutions in the dialysis membrane were collected and formulated to the solutions that had 100-fold concentration of critical micelle concentration (CMC). The particle sizes of the formulated solutions were analyzed with a particle size analyzer (COULTER, N5 Plus). The samples for particle size analyses were filtered with a 0.45 μm-filter membranes before analysis. The samples were put in a quartz tank. The temperature for analysis was set at 25° C. and the light scattering angle was set at 90°. The average particle sizes and distribution of particle sizes are recorded in Table 9.

TABLE 9

| | Theoretic Graft Ratio/ | |
|---|---|---|
| | NMR Experimental | |
| Micelle Materials | Graft Ratio (%) | Particle Size(Nm) |
| HA-g-(_{HZ}PCL₃₀₀₀-PEG₅₅₀) | 6/6 | 72.4 |
| HA-g-(_{HZ}PCL₃₀₀₀-PEG₅₅₀) | 12/13 | 83.4 |
| HA-g-(_{HZ}PCL₃₀₀₀-PEG₅₅₀) | 12/10 | 78.3 |
| HA-g-(_{HZ}PCL₃₀₀₀-PEG₅₅₀) | 20/20 | 73.9 |
| HA-g-(_{HZ}PCL₃₀₀₀-PEG₁₉₀₀) | 20/20 | 29.7 |
| HA-g-(_{HZ}PCL₃₀₀₀-PEG₁₉₀₀) | 6/5 | 30.7 |
| HA-g-(_{HZ}PCL₃₀₀₀-PEG₁₉₀₀) | 12/12 | 32.3 |
| HA-g-(_{HZ}PCL₆₀₀₀-PEG₁₉₀₀) | 4/2 | 82.3 |
| HA-g-(_{HZ}PCL₆₀₀₀-PEG₁₉₀₀) | 10/8 | 33.3 |
| HA-g-(_{HZ}PCL₁₃₅₀-PEG₅₀₀₀) | 50/50 | 101.8 |
| HA-g-(_{HZ}PCL₉₀₀-PEG₁₉₀₀) | 60/11 | 67.9 |
| HA-g-(PCL₃₀₀₀-PEG₅₅₀) | 10/13 | 138.5 |
| HA-g-(PCL₃₀₀₀-PEG₅₅₀) | 20/23 | 149.5 |
| HA-g-(PCL₃₀₀₀-PEG₁₉₀₀) | 6/7 | 159.7 |
| HA-g-(PCL₃₀₀₀-PEG₁₉₀₀) | 20/21 | 92.2 |
| HA-g-(PCL₆₀₀₀-PEG₁₉₀₀) | 10/17 | 155.2 |

### Example 11

### Acidic-Hydrolysis Test

Sample 40 of the HA-g-(_{HZ}PCL-PEG) obtained in Example 7 was formulated to five HA-g-(_{HZ}PCL-PEG) micelle aqueous solutions in equal concentrations which were 100 fold of CMC (pH 5.0). The micelle aqueous solutions were respectively added to a dialysis membrane (MWCO 6000˜8000) and dialyzed at 37° C., pH 5.0 for 24 hours. During the dialysis, the samples in the dialysis membrane were collected at different time periods, and the collected samples were immediately neutralized. After being dialyzed in DMSO for 2 days, the PEG-PCL-hydrazide was removed. The ratio of acidic-hydrolysis of the micelle formed with HA-g-(_{HZ}PCL-PEG) at pH 5.0 was calculated according to the proton signals of the hyaluronic acid (δ 1.97 ppm, s) at δ 1.2-2.5 ppm on the 1H NMR spectra. The results are shown in Table 10.

TABLE 10

| | 0 Hour | 1 Hour | 2 Hours | 6 Hours | 24 Hours |
|---|---|---|---|---|---|
| Acidic-hydrolysis | 0 | 8.2 | 56.8 | 71.2 | 71.2 |
| Ratio of _{Hz}PCL-PEG | | | | | |
| (%) | | | | | |

### Example 12

### Micelle Encapsulated Drug and Acidic Release Tests

50 mg of Sample 34 obtained in Example 7 and 2.0 mg of rhodamine-123 were added to 10 mL of DMSO. The solution was dissolved under sonication for 5 min. After being left to stand at room temperature for one day, the solution was added to a dialysis membrane (Spectrum, MWCO 3,500) and dialyzed in secondary water (pH 8.0) for two days to remove the rhodamine-123 that was not encapsulated. The remaining micelle solution encapsulating rhodamine-123 was frozen and dried.

The frozen micelle 10 mg was re-dissolved in the water and then added into a dialysis membrane (Spectrum, MWCO 3,500) for acidic-hydrolysis in double deionized water (pH 5.0). The result showed that the micelle solution encapsulating rhodamine-123 appeared orange (the original color of rhodamine-123) at Hour 0 and turned to yellow-green after 6 hours. The micelle solution appeared transparent after around 20 hours. This result showed that the rhodamine-123 encapsulated in the micelle was released in an acidic condition. Meanwhile, the samples collected at Hour 0 and Hour 6 were analyzed with ¹H NMR spectroscopy and the spectra were compared. The results show that the H signal of hydrazone at δ 9.10, 6.51 ppm and the H signal of PCL at δ 1.10-1.32 ppm are eliminated at Hour 6 compared to those at Hour 0. The results show that the hydrazone is hydrolyzed in an acidic condition, and the PEG-PCL fragment is removed by dialysis.

### Example 13

### Formulation of Doxorubicin Encapsulated With Hyaluronic Acid Derivative

2 mg of doxorubicin was mixed with 1 mL of DMSO. Triethylamine (TEA) in an equivalent concentration 3-fold higher than that of the doxorubicin was added into the solution and stirred overnight. Meanwhile, the micelle materials obtained in Examples 6 to 8 were respectively weighted to 10 mg, mixed with a co-solvent of DMSO and deionized water (v/v=2/1) 2 mL and stirring 1 hour for complete dissolution. Each of the micelle solutions were mixed with the doxorubicin solution and stirred for 0.5 hours for uniformly mixing. The mixture was then added to a dialysis membrane (MWCO=3500) and dialyzed with double deionized water or phosphate buffered saline (PBS) (pH 7.4) for one day. The samples obtained were tested for encapsulation efficiency and particle sizes.

The encapsulation efficiency and particle sizes of the formulations are shown in Tables 11 and 12. The results show that the introduction of the hydrophilic PEG chain fragments into the hydrophobic PCL chain fragments not only increase the hydrophilicity of the micelle materials but also increase the drug release in acidic conditions because the linkage (hydrazone linkage) broken in the acidic conditions causes the encapsulated drugs released from micelles.

TABLE 11

| Formulation | Sample | D/P | Aqueous | P.S. | | E.E. |
|---|---|---|---|---|---|---|
| No. | (wt %) | ratio | phase | (nm) | PI | (%) |
| 1 | 0.2 wt % HA₁₆ₖ-g-PCL_{4.4k} | 1/5 | DI water | 203.1 | 0.58 | 18.5 |
| | (PCL graft ratio: 4.6%) | | | | | |
| 2 | 0.2 wt % HA₇ₖ-g-PCL_{4.4k} | 1/5 | DI water | 146.7 | 0.48 | 19.5 |
| | (PCL graft ratio: 9.0%) | | | | | |
| 3 | 0.2 wt % HA_{16k-Hz}PCL₄ₖ | 1/5 | DI water | pptes. | — | — |
| | (obtained in Example 6) | | | | | |
| | (PCL graft ratio: 10%) | | | | | |
| 4 | 0.2 wt % HA₁₆ₖ-_{Hz}PCL₄ₖ | 1/5 | DI water | pptes. | — | — |
| | (obtained in Example 6) | | | | | |
| | (PCL graft ratio: 20%) | | | | | |
| 5 | 0.2 wt % HA₁₆ₖ-g-PCL_{4.4k} | 1/4 | DI water | 203.2 | 0.52 | 40.6 |
| | (PCL graft ratio: 4.6%) | | | | | |
| 6 | 0.2 wt % HA₇ₖ-g-PCL_{4.4k} | 1/4 | DI water | 152.4 | 0.37 | 49.4 |
| | (PCL graft ratio: 9.0%) | | | | | |
| 7 | 0.2 wt % HA₁₆ₖ-g-PCL_{4.4k} | 1/4 | PBS | 403.2 | 0.22 | 83.9 |
| | (PCL graft ratio: 4.6%) | | | | | |
| 8 | 0.2 wt % HA₇ₖ-g-PCL_{4.4k} | 1/4 | PBS | 353.3 | 0.23 | 88.6 |
| | (PCL graft ratio: 9.0%) | | | | | |

D/P ratio: a ratio of doxorubicin to micelle materials; P.S.: particle size; E.E.: encapsulation rate; pptes.: precipitates.

TABLE 12

| Formulation | Sample | D/P | P.S. | | [DXR] | E.E. |
|---|---|---|---|---|---|---|
| No. | ( wt %) | ratio | (nm) | PI | (mg/ml) | (%) |
| 9 | 0.25 wt % HA-g-(PCL-PEG) | 1/5 | 178.8 | 0.11 | 0.27 | 54.0 |
| | (Sample 42 in Example 8) | | | | | |
| 10 | 0.25 wt % HA-g-(_{Hz}PCL-PEG) | 1/5 | 150.0 | 0.16 | 0.27 | 54.0 |
| | (Sample 33 in Example 7) | | | | | |
| 11 | 0.25 wt % HA-g-(_{Hz}PCL-PEG) | 1/5 | 139.1 | 0.21 | 0.28 | 56.0 |
| | (Sample 35 in Example 7) | | | | | |
| 12 | 0.25 wt % HA-g-(_{Hz}PCL-PEG) | 1/5 | 125.9 | 0.20 | 0.27 | 54.0 |
| | (Sample 36 in Example 7) | | | | | |
| 13 | 0.33 wt % HA-g-(_{Hz}PCL-PEG) | 1/5 | 165.1 | 0.20 | 0.22 | 44.0 |
| | (Sample 32 in Example 7) | | | | | |
| 14 | 0.25 wt % HA-g-(PCL-PEG) | 1/5 | 181.4 | 0.19 | 0.23 | 46.0 |
| | (Sample 41 in Example 8) | | | | | |
| 15 | 0.25 wt % HA-g-(_{Hz}PCL-PEG) | 1/5 | 165.1 | 0.23 | 0.22 | 44.0 |
| | (Sample 38 in Example 7) | | | | | |
| 16 | 0.25 wt % HA-g-(_{Hz}PCL-PEG) | 1/5 | 178.9 | 0.38 | 0.25 | 50.0 |
| | (Sample 37 in Example 7) | | | | | |

D/P ratio: a ratio of doxorubicin to micelle materials; P.S.: particle size; E.E.: encapsulation rate.

### Example 14

### Drug Release of Doxorubicin Encapsulated with Hyaluronic Acid Derivative

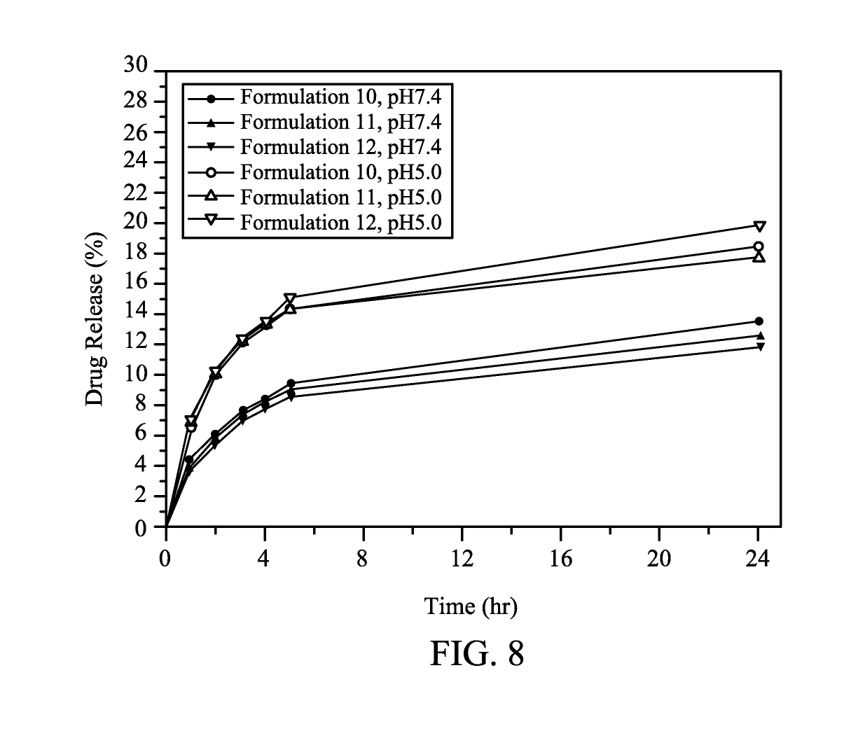

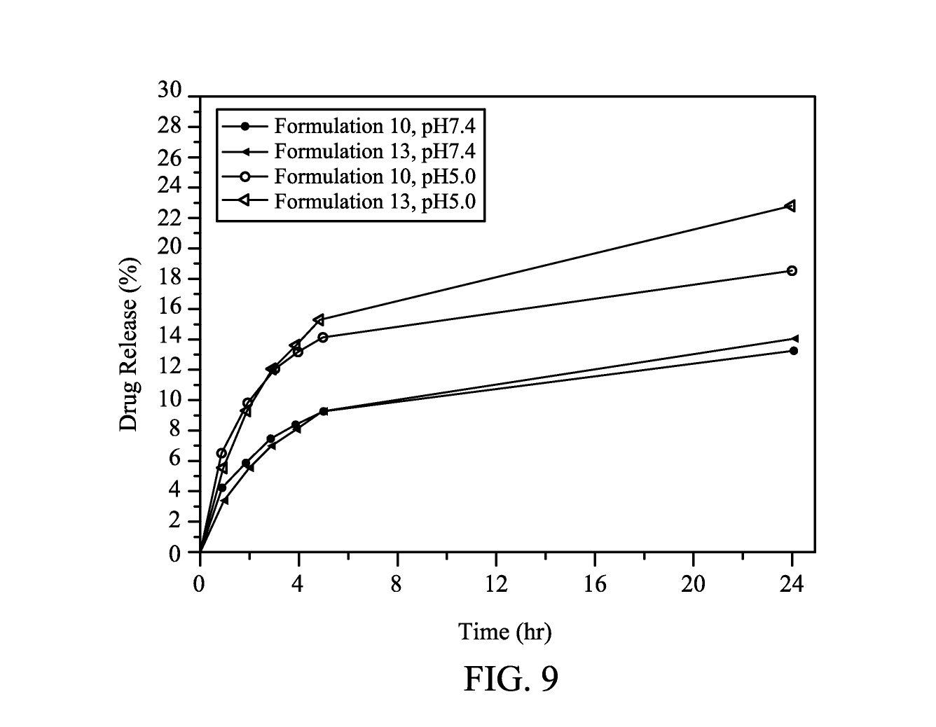

Doxorubicin was encapsulated with the hyaluronic acid derivatives following the formulations and the process described in Example 13. The release of doxorubicin was tested and recorded for 24 hours as shown in FIGS. 8 and 9. FIG. 8 shows the release profile of doxorubicin encapsulated with HA-g-(_{Hz}PCL-PEG) obtained in Example 13 at various pH values. The results show that the release of doxorubicin in acidic condition is about 2-fold higher than that in neutral conditions. The results reveal that the linkage (hydrazone) caused the increased release of doxorubicin in acidic conditions. FIG. 9 shows the effects of the chain lengths of HA-g-(_{Hz}PCL-PEG) obtained in Example 13 on drug release. Results show that various hydrophilic chain (PEG) lengths show a pH-sensitive property due to the linkage (hydrazone linkage). In addition, the results show that the release of doxorubicin is increased when the hydrophilic chain (PEG) length is longer.

It will be apparent to those skilled in the art that various modifications and variations can be made to the disclosed embodiments. It is intended that the specification and examples be considered as exemplary only, with a true scope of the disclosure being indicated by the following claims and their equivalents.

1. A hyaluronic acid derivative, comprising a structure as shown in the following formula (I): 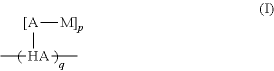
In the formula,
HA represents a unit comprising N-acetyl-D-glucosamine and D-glucuronic acid, q represents an integer of 2 to 10,000;
A represents a biocleavable linkage comprising at least one of hydrazone, acetal, ketal and imine;
M represents at least one of a hydrophobic fragment, a hydrophilic fragment, and an amphiphilic fragment, and
p represents the number of [A-M] directly grafted onto each HA unit, p represents an integer of 0 to 4, and the p of each HA unit is not 0 at the same time;
wherein the hyaluronic acid derivative is biodegradable and pH-sensitive, and linkages of the hyaluronic acid derivative are broken in acidic conditions.

2. The hyaluronic acid derivative as claimed in claim 1, wherein the biocleavable linkage is hydrolyzed in acidic conditions.

3. The hyaluronic acid derivative as claimed in claim 1, wherein the biocleavable linkage forms covalent connection with at least one hydroxyl group of the N-acetyl-D-glucosamine and D-glucuronic acid.

4. The hyaluronic acid derivative as claimed in claim 1, wherein the hydrophobic fragment consists of repeat units of a bioabsorbable polymer.

5. The hyaluronic acid derivative as claimed in claim 4, wherein the repeat units of bioabsorbable polymer comprises caprolactone, butyrolactone, D-lactide, L-lactide, D-lactic acid, L-lactic acid, glycolide, glycolic acid, hydroxyl hexonoic acid, hydroxyl butyric acid, valerolactone, hydroxyl valeric acid, malic acid, a copolymer thereof or a combination thereof.

6. The hyaluronic acid derivative as claimed in claim 1, wherein the amphiphilic fragment consists of a hydrophobic region and a hydrophilic region.

7. The hyaluronic acid derivative as claimed in claim 6, wherein the hydrophobic region includes a repeated unit comprising caprolactone, butyrolactone, D-lactide, L-lactide, D-lactic acid, L-lactic acid, glycolide, glycolic acid, hydroxyl hexonoic acid, hydroxyl butyric acid, valerolactone, hydroxy valeric acid, malic acid, a copolymer thereof or a combination thereof.

8. The hyaluronic acid derivative as claimed in claim 6, wherein the hydrophilic region comprises a repeated unit comprising ethylene glycol, ethylene oxide, vinylpyrrolidone, acrylic acid, methacrylic acid, a copolymer thereof or a combination thereof.

9. The hyaluronic acid derivative as claimed in claim 1, wherein the hydrophobic fragment or the amphiphilic fragment comprise at least one linkage that is broken in acidic conditions.

10. The hyaluronic acid derivative as claimed in claim 9, wherein the linkage that is broken in acidic conditions comprises at least one of hydrazone, acetal, ketal and imine.

11. The hyaluronic acid derivative as claimed in claim 1, wherein the hydrophilic fragment comprises polyethylene glycol (PEG), polyethylene oxide (PEO), polyvinylpyrrolidone (PVP), polyacrylic acid (PAA), polymethacrylic acid (PMA), or combination thereof.

12. The hyaluronic acid derivative as claimed in claim 1, wherein the M has a molecular weight of 100 to 50,000 Da.

13. The hyaluronic acid derivative as claimed in claim 1, further comprising a hydrophilic fragment grafting to the HA with a non-biocleavable linkage or a linkage that is not easily or quickly broken.

14. The hyaluronic acid derivative as claimed in claim 13, wherein the hydrophilic fragment comprises repeat units comprising ethylene glycol, ethylene oxide, vinylpyrrolidone, acrylic acid, methacrylic acid, a copolymer thereof or a combination thereof.

15. The hyaluronic acid derivative as claimed in claim 13, wherein the linkage that is not easily or quickly broken comprises an ester bond.

16. The hyaluronic acid derivative as claimed in claim 13, wherein the non-biocleavable linkage comprises urethane linkage.

17. The hyaluronic acid derivative as claimed in claim 1, further comprising a protein molecule grafting to the HA with an isolated chain fragment, wherein the protein molecule is bio-identifiable.

18. The hyaluronic acid derivative as claimed in claim 17, wherein the protein molecule comprises an antibody or ligand.

19. A micelle, formed by the hyaluronic acid derivative of claim 1 in a hydrophilic medium.

20. The micelle as claimed in claim 19, wherein the hyaluronic acid derivative is 0.0001 to 10% by weight based on the weight of the hydrophilic medium.

21. A drug delivery system, comprising a carrier and a bioactive ingredient encapsulated with the carrier, wherein the carrier consists of the hyaluronic acid derivative of claim 1.

22. The drug delivery system as claimed in claim 21, wherein the carrier forms a structure of micelles.

23. The drug delivery system as claimed in claim 21, wherein the bioactive ingredient comprises drugs or nutrients.

24. The drug delivery system as claimed in claim 21, wherein the bioactive ingredient comprises anticancer drugs.

25. The drug delivery system as claimed in claim 21, wherein the bioactive ingredient and the hyaluronic acid derivative have a weight ratio of 1:1 to 1:20.

26. A flavor enhancer, for encapsulating a bioactive material to reduce the flavor thereof, wherein the flavor enhancer consists of the hyaluronic acid derivative of claim 1.
